(19) United States
(12) Patent Application Publication
(10) Pub. No.: US 2022/0249118 A1
(43) Pub. Date: Aug. 11, 2022

(54) SURGICAL SLICING SHEARS

(71) Applicant: Covidien LP, Mansfield, MA (US)

(72) Inventors: Justin J. Thomas, New Haven, CT (US); Roy J. Pilletere, Middletown, CT (US); Jacob C. Baril, Norwalk, CT (US); Matthew A. Dinino, Newington, CT (US); Nicolette L. Roy, Windsor Locks, CT (US); Saumya Banerjee, Collinsville, CT (US); Garrett P. Ebersole, Hamden, CT (US)

(21) Appl. No.: 17/582,543

(22) Filed: Jan. 24, 2022

Related U.S. Application Data
(60) Provisional application No. 63/147,437, filed on Feb. 9, 2021.

(51) Int. Cl. A61B 17/32; A61B 17/3201

(52) U.S. Cl. CPC A61B 17/320016; A61B 17/3201

(57) ABSTRACT

An end effector for a surgical device includes first and second jaws disposed adjacent one another. An actuator clevis is configured to couple to proximal ends of the first and second jaws in a distal portion of the actuator clevis. A jaw clevis defines a lumen and is configured to receive the first and second jaws and the actuator clevis in the lumen. The first and second jaws and the actuator clevis are configured to be transitionable along a longitudinal axis of the lumen. The first and second jaws are pivotably coupled to each other at proximal ends thereof and slidably coupled to the jaw clevis at a location distal of the proximal ends.

### CROSS-REFERENCE TO RELATED APPLICATIONS

The present application claims the benefit of, and priority to, U.S. Provisional Patent Application Ser. No. 63/147,437, filed Feb. 9, 2021, the entire contents of which are incorporated by reference herein.

### FIELD

The present disclosure is generally related to surgical instruments. More particularly, the present disclosure relates to a slicing shear instrument for slicing and cutting tissue.

### BACKGROUND

During surgery, it is often necessary to cut or otherwise sever a tissue. Typically, a pair of surgical shears or scissors are used to cut tissue such as a tumor, vessel, muscle, etc. Surgical shears cut tissue by placing two opposing blades in interference with one another, rubbing at the cutting edge to dissect tissue. However, shear blades wear and dull relatively quickly after making multiple cuts in this manner, requiring a new set of shears to be used. A dull blade can cause excessive damage to a tissue or introduce tears in the tissue beyond that which is intended or desired, and in some cases, a dull blade may not even fully sever tissue as intended. Thus, surgical shears are often replaced during surgical procedures even after only a few cuts may have been made to stay within a preferred ‘sharpness range’ of a pair of surgical shears. This leads to an increase in the duration and cost of the surgical procedure.

### SUMMARY

This disclosure generally relates to an end effector for a surgical device. The end effector includes first and second jaws disposed adjacent one another. An actuator clevis is configured to couple to proximal ends of the first and second jaws in a distal portion of the actuator clevis. The jaw clevis defines a lumen and is configured to receive the first and second jaws and the actuator clevis in the lumen. The first and second jaws and the actuator clevis are configured to be transitionable along a longitudinal axis of the lumen. The first and second jaws are pivotably coupled to each other at proximal ends thereof and slidably coupled to the jaw clevis at a location distal of the proximal ends of the first and second jaws.

In aspects, a distal position of the actuator clevis and the first and second jaws may define an open configuration and a proximal position of the actuator clevis and the first and second jaws may define an approximated configuration, the first and second jaws transitionable between the open and approximated configurations.

In aspects, as the first and second jaws transition from the open configuration to the approximated configuration, the first and second jaws may simultaneously cut and slice an object disposed between the first and second jaws.

In other aspects, as the first and second jaws transition from the open configuration to the approximated configuration, the first and second jaws may pivot about the jaw clevis in a cutting motion and may move proximally in a slicing motion approximately simultaneously.

In yet further aspects, a proximal portion of the actuator clevis may be configured to couple to an actuation mechanism.

In some aspects, the actuation mechanism may be configured to transition the first and second jaws between the open and approximated configurations.

In more aspects, the actuation mechanism may include an actuator shaft coupled to the proximal portion of the actuator clevis, and an outer tube coupled to a proximal portion of the jaw clevis.

In alternative aspects, the actuator shaft may be disposed in the outer tube. The outer tube may be configured to extend the lumen of the jaw clevis along the longitudinal axis such that the first and second jaws and actuator clevis are transitionable distally and proximally along the longitudinal axis.

In aspects, the first and second jaws may include slots configured to receive a jaw clevis pin, the first and second jaws may be pivotably and slidably coupled to the jaw clevis about the jaw clevis pin.

In further aspects, the slots of the first and second jaws may be configured to permit the first and second jaws to pivot about the jaw clevis pin to facilitate a cutting motion and the slots may be configured to permit proximal movement of the first and second jaws to facilitate a slicing motion, such that the incremental cutting motion is accompanied by incremental slicing motion.

In yet further aspects, the first and second jaws may include slots configured to receive a jaw clevis pin, wherein the first and second jaws are coupled to the jaw clevis about the jaw clevis pin.

In some other aspects, the slots of the first and second jaws may be configured to permit the first and second jaws to pivot about the jaw clevis pin to facilitate a cutting motion and the slots may be configured to permit proximal movement of the first and second jaws to facilitate a slicing motion.

In even further aspects, the jaw clevis pin may be disposed in a proximal portion of the slots of the first and second jaws in the open configuration and is disposed in a distal portion of the slots of the first and second jaws in the approximated configuration.

In aspects, the first and second jaws may include blades manufactured from stainless steel, titanium, ceramic, or toughened resins.

This disclosure also relates to a surgical device for separating tissue. The surgical device includes an actuator shaft, a jaw clevis, an actuator clevis, and first and second jaws. The actuator clevis is disposed at a distal portion of the actuator shaft and is configured to be received by the jaw clevis. The actuator clevis and the actuator shaft are configured to move along a longitudinal axis of the jaw clevis. The first and second jaws are disposed at a distal portion of the actuator clevis. The first and second jaws are pivotably coupled to each other and the actuator clevis at proximal ends thereof, and the first and second jaws are pivotably and slidably coupled to the jaw clevis at a location distal of the proximal ends thereof. The first and second jaws are transitionable between an open configuration and an approximated configuration, the approximated configuration defined by the actuator shaft, the actuator clevis, and the first and second jaws being in a proximal position and the open configuration defined by the actuator shaft, the actuator clevis, and the first and second jaws being in a distal position.

In aspects, the first and second jaws may be transitionable from the open configuration to the approximated configuration such that the first and second jaws approximately simultaneously slice and cut an object therebetween.

In other aspects, the first and second jaws may be transitionable from the open configuration to the approximated configuration such that the first and second jaws move into and across an object therebetween.

In another aspect, an actuation mechanism may be coupled to the actuator shaft and configured to transition the first and second jaws between the open and approximated configurations.

In some aspects, the surgical device may further include an outer tube, wherein the jaw clevis is coupled to a distal portion of the outer tube, the outer tube and jaw clevis defining a lumen configured to receive the first and second jaws and the actuator clevis.

This disclosure additionally relates to an end effector for severing tissue. The end effector includes: an actuator shaft, a jaw clevis, and first and second jaws. The first and second jaws are pivotably and slidably coupled to the jaw clevis at a location distal of proximal ends of the first and second jaws. An actuator is coupled to proximal ends of the first and second jaws, the actuator configured to transition the first and second jaws between an open configuration and an approximated configuration. The approximated configuration is defined by the proximal ends of the first and second jaws being in a proximal position and distal ends of the first and second jaws being approximately adjacent each other. The open configuration is defined by the proximal ends of the first and second jaws being in a distal position and the distal ends of the first and second jaws are spaced apart from each other. As the first and second jaws transition from the open configuration to the approximated configuration, the first and second jaws slice and cut an object therebetween approximately simultaneously.

The details of one or more aspects of the disclosure are set forth in the accompanying drawings and the description below. Other features, objects, and advantages of the techniques described in this disclosure will be apparent from the description and drawings, and from the claims.

### DESCRIPTION OF DRAWINGS

The accompanying drawings, which are incorporated in and constitute a part of this specification, illustrate aspects and features of the disclosure and, together with the detailed description below, serve to further explain the disclosure, in which:

FIG. 1 is a perspective view of a slicing shears instrument including an end effector with first and second jaws with an actuator mechanism shown in phantom;

FIG. 2 is an enlarged view of the area of detail identified in FIG. 1 illustrating the end effector of the slicing shears;

FIG. 3 is an internal view of the area of detail shown in FIG. 2, with a jaw clevis made transparent and shown in phantom;

FIG. 4 is an exploded perspective view, with parts separated, of the end effector shown in FIG. 2;

FIG. 5 is a perspective, side view of a slicing shears instrument with the first and second jaws in an open configuration and positioned about a tissue;

FIG. 6 is a side view of the end effector of FIG. 2 with the first and second jaws in an open configuration;

FIG. 7 is a side view of the end effector of FIG. 2 with the first and second jaws in an approximated configuration;

Further details and various aspects of this disclosure are described in more detail below with reference to the appended figures.

### DETAILED DESCRIPTION

Aspects of the presently disclosed slicing shears instrument are described in detail with reference to the drawings, in which like reference numerals designate identical or corresponding elements in each of the several views. However, it is to be understood that the disclosed devices are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

Descriptions of technical features of an illustrative slicing shears instrument in accordance with the disclosure should typically be considered as available and applicable to other similar features of another device of the disclosure. Accordingly, technical features described herein in connection with one illustrative slicing shears instrument may be applicable to other devices of the disclosure, and thus duplicative descriptions may be omitted herein.

As used herein, the term “distal” refers to that portion of the surgical instrument, or component thereof, farther from the user, while the term “proximal” refers to that portion of the surgical instrument, or component thereof, closer to the user.

As used herein, the term “approximated configuration” refers to a configuration where the jaws of the end effector of the slicing shears instrument are in an approximately parallel configuration. The term “open configuration” refers to a configuration where the jaws of the end effector of the slicing shears instrument form an angle wherein the jaws are capable of receiving an object (e.g., tissue) therebetween.

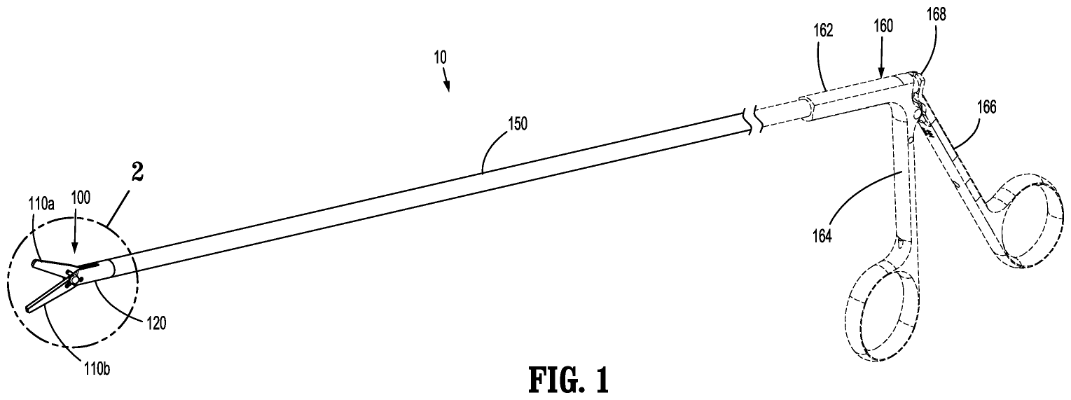

Referring to FIG. 1, a slicing shears instrument 10 including an end effector 100 with a first jaw 110a, a second jaw 110b, an outer tube 150, and an actuation mechanism or handle assembly 160 is shown. The end effector 100 is disposed at a distal end of the outer tube 150, and the handle assembly 160 is disposed at a proximal end of the outer tube 150. The handle assembly 160 includes a body 162, a stationary handle 164, a trigger 166, and a collar 168 for coupling the end effector 100 to the handle assembly 160 via an actuator shaft 140. The end effector 100 is configured to receive, and approximately simultaneously slice and cut a section of tissue or another object desired to be severed or cut. The end effector 100 may be operated manually by pivoting the trigger 166 of the handle assembly 160 towards the stationary handle 164. Alternatively, a robotic actuation mechanism (not shown) may be employed to actuate the end effector 100. In various aspects, the robotic actuation mechanism may be controlled by a surgeon via a computing device in electrical communication with the actuation mechanism. The end effector 100 may be operated by a computing device, server, and/or network (not shown), which may include memory, storage device(s), controllers (e.g. software) for operating the end effector.

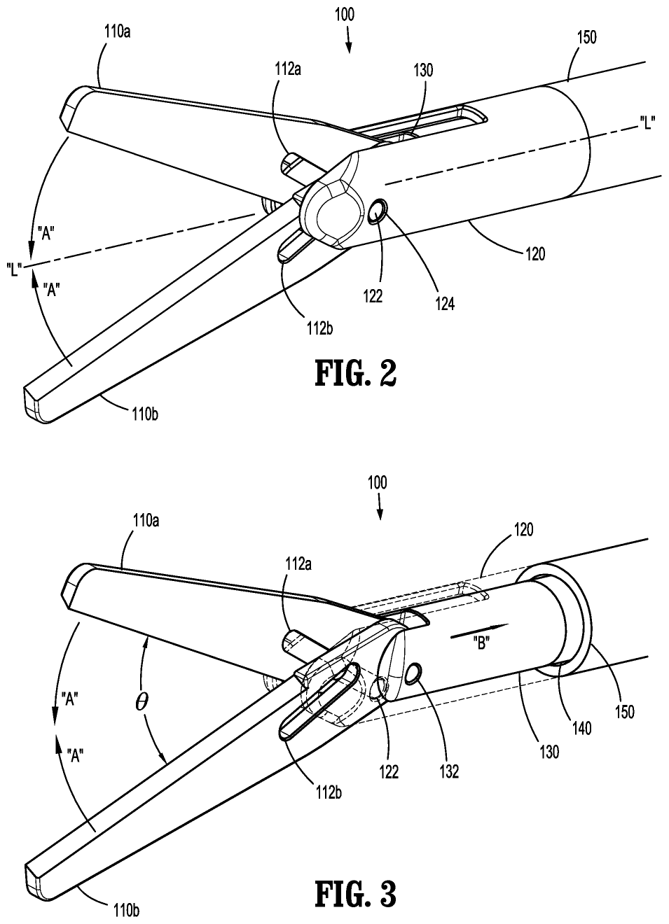

With reference to FIGS. 2 and 3, the end effector 100 with first and second jaws 110a, 110b is shown in further detail. The end effector 100 is configured such that the first and second jaws 110a, 110b cooperate to define a cutting motion in the direction indicated by arrows “A” (FIGS. 2 & 6) while the first and second jaws 110a, 110b also move proximally as indicated by arrows “B” (FIGS. 3 & 7).

The end effector 100 includes a jaw clevis 120, an actuator clevis 130, and an actuator shaft 140. The first and second jaws 110a, 110b are pivotably coupled at proximal ends thereof to a distal portion of the actuator clevis 130. The first and second jaws 110a, 110b are also pivotably and slidably coupled to the jaw clevis 120 at a location distal of proximal ends of the first and second jaws 110a, 110b. The actuator clevis 130 is coupled at a proximal portion thereof to a distal portion of the actuator shaft 140. Actuator shaft 140 may be a hollow tube, rod, or other suitable rigid members.

The jaw clevis 120 is coupled at a proximal portion thereof to a distal portion of the outer tube 150. The outer tube 150 and the jaw clevis 120 may be co-axial along a longitudinal axis “L” thereof, the outer tube 150 and the jaw clevis 120 each defining a lumen. The actuator clevis 130 and first and second jaws 110a, 110b are disposed in the lumen of the jaw clevis 120. The actuator shaft 140 is disposed within the lumen of the outer tube 150. In operation, the first and second jaws 110a, 110b and actuator clevis 130 may be configured to move proximally and distally within the lumens of the jaw clevis 120 and the outer tube 150 and along the longitudinal axis “L.”

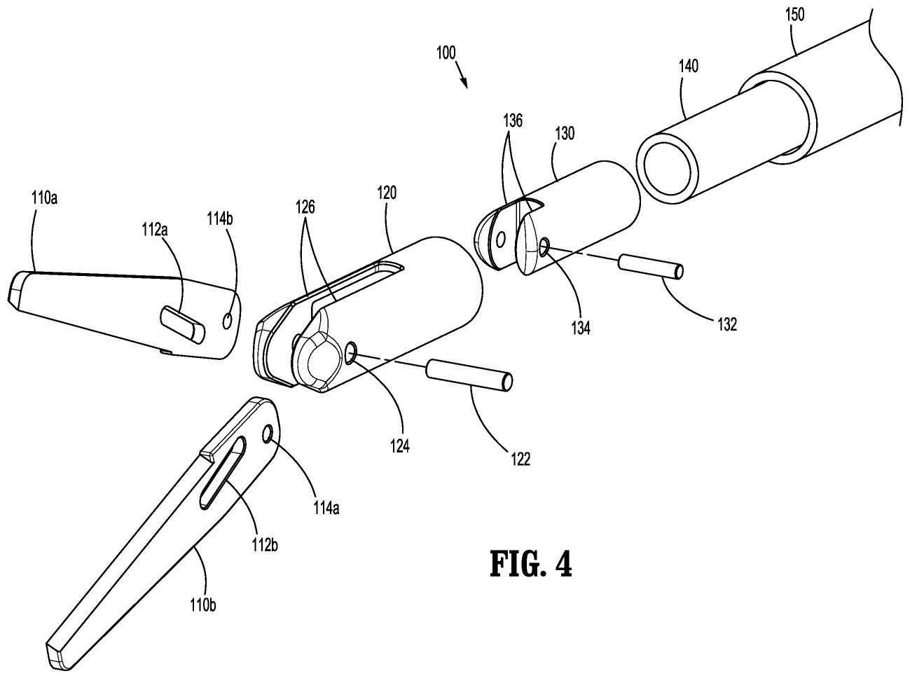

With additional reference to FIG. 4, the first and second jaws 110a, 110b are pivotably coupled to each other and the actuator clevis 130 at their proximal ends via an actuator pin 132. First and second jaws 110a, 110b include slots 112a, 112b, respectively. Slots 112a, 112b are configured to receive jaw clevis pin 122. Jaw clevis pin 122 is also received by hole 124 of jaw clevis 120, such that, when assembled, jaw clevis pin 122 is configured to couple first and second jaws 110a, 110b to jaw clevis 120. The slots 112a, 112b are configured to permit the first and second jaws 110a, 110b to slide over and rotate about jaw clevis pin 122. First and second jaws 110a, 110b are also pivotably coupled to actuator clevis 130 via actuator clevis pin 132 at proximal ends of the first and second jaws 110a, 110b. First and second jaws 110a, 110b are configured to rotate about the actuator clevis pin 132.

The jaw clevis 120 and the actuator clevis 130 include prongs 126 and 136, respectively, that define u-shaped slots that are co-planar and configured such that the first and second jaws 110a, 110b may rotate about the jaw clevis pin 122 and actuator pin 132 without being constrained due to misalignment of the jaw clevis 120 or actuator clevis 130. The first and second jaws 110a, 110b are positioned between the prongs 126, 136 of the jaw clevis 120 and actuator clevis 130, respectively, and the jaw clevis pin 122 is inserted into holes 124 and slots 112a, 112b, and the actuator clevis pin 132 is inserted into holes 134 of the actuator clevis and holes 114a, 114b of the first and second jaws 110a, 110b.

Slots 112a, 112b may be shaped to define a maximum angle θ (FIGS. 3 & 6) between the first jaw 110a and second jaw 110b when the end effector 100 is in an open configuration. For example, when the first and second jaws 110a, 110b are positioned such that the jaw clevis pin 122 is in the proximal-most position within slots 112a, 112b, the angle θ formed between the first and second jaws 110a, 110b is at a maximum. The maximum angle θ between the first and second jaws 110a, 110b may be about 90 degrees such that each jaw may rotate up to about 45 degrees relative to the longitudinal axis “L”. In aspects, the maximum angle θ is 45 degrees, such that each jaw defines a 22.5 degree angle with respect to the longitudinal axis “L.” It is contemplated that the maximum angle θ may be greater than 90 degrees or less than 45 degrees without departing from the scope of this disclosure. When the jaw clevis pin 122 is in a distal-most position in slots 112a, 112b, the angle θ between jaws 110a, 110b is negligible (e.g., approximately zero), such that the distal ends of the first and second jaws 110a, 110b are approximately adjacent (see FIG. 7). While slots 112a, 112b are illustrated as linear slots, any suitable slot geometry configured to allow the first and second jaws 110a, 110b to pivot and slide about the jaw clevis pin 122 may be used, such as a “j” shaped slot.

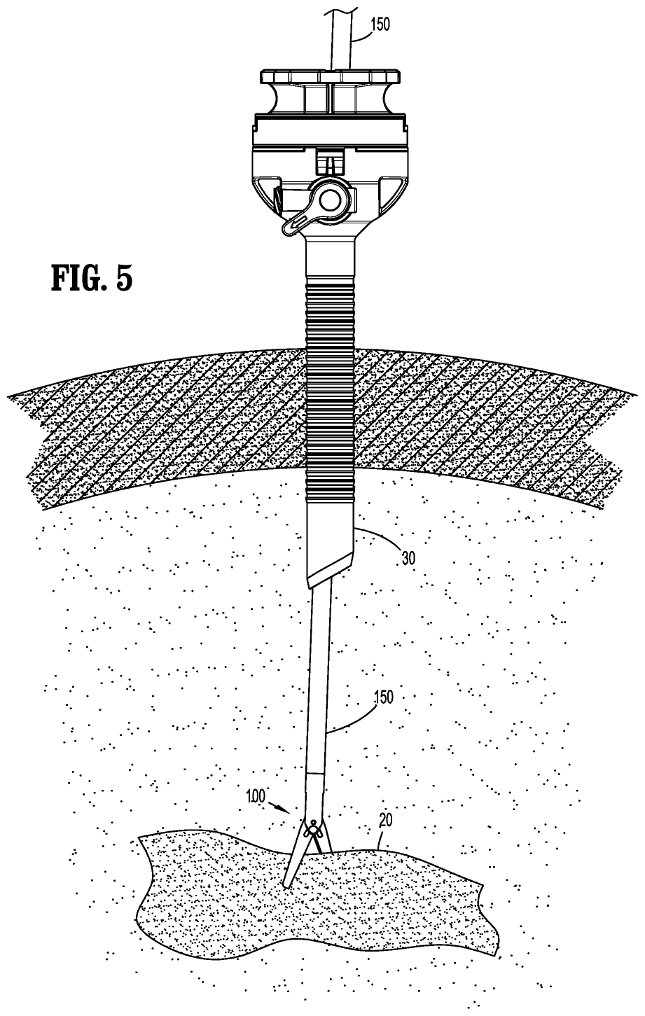

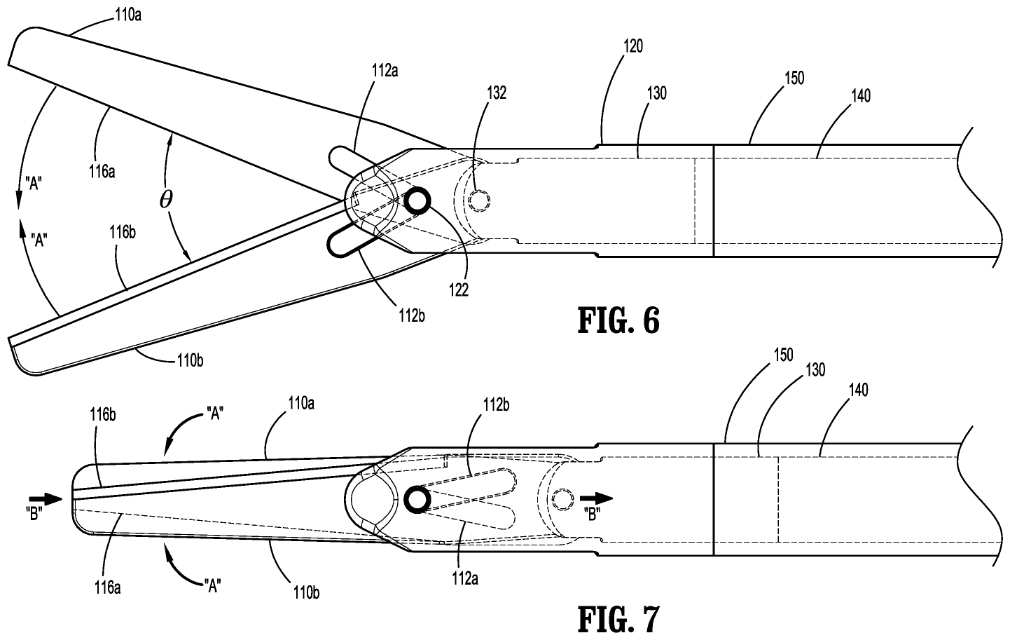

With reference to FIGS. 5, 6, and 7, the end effector 100 is configured to produce a cutting and slicing motion such that tissue may be cut and sliced simultaneously. As illustrated in FIG. 5, the end effector 100 of the slicing shears instrument 10 may be inserted through a surgical access device 30 to sever a tissue 20. The first and second jaws 110a, 110b include blades 116a, 116b, respectively, and are positioned in an open configuration about the tissue to be severed. The actuation mechanism or handle assembly 160 (shown in FIG. 1) controls the position of the first and second jaws 110a, 110b allowing a user to transition the end effector 100 from the open configuration (FIG. 6) to the approximated configuration (FIG. 7) and vice versa.

In operation, the first and second jaws 110a, 110b are used to slice and cut tissue, such that incremental cutting motion of the first and second jaws 110a, 110b is accompanied by incremental slicing motion of the first and second jaws 110a, 110b. Thus, the first and second jaws 110a, 110b are able to approximately simultaneously slice and cut tissue. In aspects, the first and second jaws 110a, 110b may be separately actuated such that one or both blades may simultaneously follow a slice and cut motion to sever tissue therebetween. The approximately simultaneous cutting and slicing motions, indicated by arrows “A” and “B” respectively, reduces wear on the blades 116a, 116b of the first and second jaws 110a, 110b, respectively, by adding the slicing motion to the cutting motion of a typical pair of surgical scissors or shears. Slicing motion introduces less wear since less pressure may be required to sever the tissue at the point of contact between the blade and the tissue. In aspects, the blades 116a, 116b of the first and second jaws 110a, 110b may be made from at least one of stainless steel, titanium, ceramic material, or toughened resins. Thus the slicing shears instrument 10 or the end effector 100 is configured to wear or dull at a slower rate compared to a standard pair of surgical scissors or shears, which may save time during a surgical procedure and lower the cost of a procedure since fewer instruments may need to be used or replaced during an operation.

The approximately simultaneous slicing and cutting motions are achieved by transitioning the end effector 100 from the open configuration to the approximated configuration. As shown in FIG. 6, in the open configuration, the jaw clevis pin 122 is in a proximal position in slots 112a, 112b of the first and second jaws 110a, 110b. The distal-most ends of the first and second jaws 110a, 110b are spaced apart from one another in the open configuration. Angle θ is at a maximum when the distal-most ends of the first and second jaws 110a, 110b are spaced apart a maximum distance, the jaw clevis pin 122 is in a proximal-most position in slots 112a, 112b, or both. The actuator clevis 130 and the first and second jaws 110a, 110b are in a distal position in the open configuration. In the closed configuration,

The first and second jaws 110a, 110b are coupled to the actuator clevis 130 such that the positions of the first and second jaws 110a, 110b along the longitudinal axis “L” relative to the distal portion of actuator clevis 130 do not change between the open configuration and the closed configuration. The first and second jaws 110a, 110b are coupled to the jaw clevis 120 such that the positions of the first and second jaws 110a, 110b relative to the distal end of the jaw clevis 120 do change between the open configuration and the approximated configuration.

In transitioning to the closed configuration, the actuator clevis 130, the first and second jaws 110a, 110b, and the actuator shaft 140 move proximally within the lumens of the jaw clevis 120 and the outer tube 150 as indicated by arrows “B”. The angle θ between the first and second jaws 110a, 110b decreases as the actuator shaft 140, and thus the actuator clevis 130, and first and second jaws 110a, 110b, move proximally. The slots 112a, 112b, as described above, force the first and second jaws 110a, 110b to rotate about the jaw clevis pin 122 to follow the cutting motion indicated by arrows “A” and slide proximally over the jaw clevis pin 122 to follow the slicing motion indicated by arrows “B.” The first and second jaws 110a, 110b are configured to rotate in the direction indicated by arrows “A” towards the approximated configuration. Thus the first and second jaws 110a, 110b move into and across a tissue or object therebetween. As the first and second jaws 110a, 110b move proximally towards the approximated configuration, the distal-most ends of the first and second jaws 110a, 110b approach each other, such that when in the approximated configuration, the distal-most ends of the first and second jaws 110a, 110b are approximately adjacent one another, and the angle θ is negligible. In the approximated configuration, the actuator clevis 130 and the first and second jaws 110a, 110b are in a proximal-most position along longitudinal axis “L.” The jaw clevis pin 122 is in a distal-most position in the slots 112a, 112b of the first and second jaws 110a, 110b. The end effector 100 may be transitioned back to the open configuration, reversing the changes in position between the relative parts thereof just described.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An end effector for a surgical device comprising:
first and second jaws disposed adjacent one another;
an actuator clevis configured to couple to proximal ends of the first and second jaws in a distal portion of the actuator clevis; and
a jaw clevis defining a lumen and configured to receive the first and second jaws and the actuator clevis in the lumen, the first and second jaws and the actuator clevis configured to be transitionable along a longitudinal axis of the lumen,
wherein the first and second jaws are pivotably coupled to each other at proximal ends thereof and slidably coupled to the jaw clevis at a location distal of the proximal ends of the first and second jaws.

2. The end effector of claim 1, wherein a distal position of the actuator clevis and the first and second jaws defines an open configuration and a proximal position of the actuator clevis and the first and second jaws defines an approximated configuration, the first and second jaws transitionable between the open and approximated configurations.

3. The end effector of claim 2, wherein as the first and second jaws transition from the open configuration to the approximated configuration, the first and second jaws simultaneously cut and slice an object disposed between the first and second jaws.

4. The end effector of claim 2, wherein as the first and second jaws transition from the open configuration to the approximated configuration, the first and second jaws pivot about the jaw clevis in a cutting motion and move proximally in a slicing motion approximately simultaneously.

5. The end effector of claim 1, wherein a proximal portion of the actuator clevis is configured to couple to an actuation mechanism.

6. The end effector of claim 5, wherein the actuation mechanism is configured to transition the first and second jaws between open and approximated configurations.

7. The end effector of claim 6, wherein the actuation mechanism includes an actuator shaft coupled to the proximal portion of the actuator clevis, and an outer tube coupled to a proximal portion of the jaw clevis.

8. The end effector of claim 7, wherein the actuator shaft is disposed in the outer tube, the outer tube configured to extend the lumen of the jaw clevis along the longitudinal axis such that the first and second jaws and actuator clevis are transitionable distally and proximally along the longitudinal axis.

9. The end effector of claim 1, wherein the first and second jaws include slots configured to receive a jaw clevis pin, the first and second jaws are pivotably and slidably coupled to the jaw clevis about the jaw clevis pin.

10. The end effector of claim 9, wherein the slots of the first and second jaws are configured to permit the first and second jaws to pivot about the jaw clevis pin to facilitate an incremental cutting motion and the slots are configured to permit proximal movement of the first and second jaws to facilitate an incremental slicing motion, such that the incremental cutting motion is accompanied by incremental slicing motion.

11. The end effector of claim 2, wherein the first and second jaws include slots configured to receive a jaw clevis pin, wherein the first and second jaws are coupled to the jaw clevis about the jaw clevis pin.

12. The end effector of claim 11, wherein the slots of the first and second jaws are configured to permit the first and second jaws to pivot about the jaw clevis pin to facilitate a cutting motion and the slots are configured to permit proximal movement of the first and second jaws to facilitate a slicing motion.

13. The end effector of claim 11, wherein the jaw clevis pin is disposed in a proximal portion of the slots of the first and second jaws in the open configuration and is disposed in a distal portion of the slots of the first and second jaws in the approximated configuration.

14. The end effector of claim 1, wherein the first and second jaws include blades manufactured from stainless steel, titanium, ceramic, or toughened resins.

15. A surgical device for separating tissue comprising:
an actuator shaft;
a jaw clevis;
an actuator clevis disposed at a distal portion of the actuator shaft and configured to be received by the jaw clevis, the actuator clevis and the actuator shaft configured to move along a longitudinal axis of the jaw clevis; and
first and second jaws disposed at a distal portion of the actuator clevis, the first and second jaws pivotably coupled to each other and the actuator clevis at proximal ends thereof, the first and second jaws pivotably and slidably coupled to the jaw clevis at a location distal of the proximal ends thereof;
wherein the first and second jaws are transitionable between an open configuration and an approximated configuration, the approximated configuration defined by the actuator shaft, the actuator clevis, and the first and second jaws being in a proximal position and the open configuration defined by the actuator shaft, the actuator clevis, and the first and second jaws being in a distal position.

16. The surgical device of claim 15, wherein first and second jaws are transitionable from the open configuration to the approximated configuration such that the first and second jaws approximately simultaneously slice and cut an object therebetween.

17. The surgical device of claim 15, wherein the first and second jaws are transitionable from the open configuration to the approximated configuration such that the first and second jaws move into and across an object therebetween.

18. The surgical device of claim 15, further including an outer tube, wherein the jaw clevis is coupled to a distal portion of the outer tube, the outer tube and jaw clevis defining a lumen configured to receive the first and second jaws and the actuator clevis.

19. The surgical device of claim 15, further including an actuation mechanism coupled to the actuator shaft and configured to transition the first and second jaws between the open and approximated configurations.

20. An end effector for severing tissue comprising:
a jaw clevis;
first and second jaws pivotably and slidably coupled to the jaw clevis at a location distal of proximal ends of the first and second jaws; and
an actuator coupled to proximal ends of the first and second jaws, the actuator configured to transition the first and second jaws between an open configuration and an approximated configuration;
wherein, the approximated configuration is defined by the proximal ends of the first and second jaws being in a proximal position and distal ends of the first and second jaws being approximately adjacent each other, and the open configuration is defined by the proximal ends of the first and second jaws being in a distal position and the distal ends of the first and second jaws are spaced apart from each other;
wherein, as the first and second jaws transition from the open configuration to the approximated configuration, the first and second jaws approximately simultaneously slice and cut an object therebetween.
